(19) United States
(12) Patent Application Publication
(10) Pub. No.: US 2024/0394408 A1
(43) Pub. Date: Nov. 28, 2024

(54) SYSTEMS AND METHODS FOR ANONYMIZATION OF IMAGE DATA

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ross Schmidtlein, New York, NY (US); Daniel Lafontaine, Flushing, NY (US)

(21) Appl. No.: 18/696,891

(22) PCT Filed: Sep. 28, 2022
(86) PCT No.: PCT/US22/45122

§ 371 Date: Mar. 28, 2024

Related U.S. Application Data
(60) Provisional application No. 63/249,896, filed on Sep. 29, 2021.

(51) Int. Cl. G06F 21/62; G06V 10/22; G06V 10/25; G06V 10/44; G06V 40/16

(52) U.S. Cl. CPC G06F 21/6254; G06V 10/235; G06V 10/25; G06V 10/44; G06V 40/168; G06V 2201/03

(57) ABSTRACT

Described embodiments provide systems and methods for anonymizing image data. A computing system can obtain a medical image of a subject, the medical image comprising a set of slices and being associated with a set of metadata regarding the medical image and the subject. The computing system may identify, based on the set of metadata, one or more regions of interest (ROIs) of the subject in the medical image, the ROIs corresponding with a condition to be evaluated by a clinician. The computing system may select, based on the ROIs, a modification technique to apply to the medical image, wherein selecting the modification technique comprises determining an image segment that is situated outside of the identified ROIs, the image segment comprising a distinguishing feature of the subject. The computing system may generate a modified image by applying the modification technique to the medical image to render the distinguishing feature indistinguishable.

### CROSS-REFERENCE TO RELATED APPLICATIONS

This patent application claims priority to and the benefit of U.S. Provisional Patent Application 63/249,896 filed Sep. 29, 2021, the contents of which are incorporated herein by reference in its entirety.

### FIELD OF THE DISCLOSURE

The present disclosure is directed to anonymizing and/or de-identifying an image of a subject by applying a modification technique to the image.

### BACKGROUND OF THE DISCLOSURE

The following description of the background of the present technology is provided simply as an aid in understanding the present technology and is not admitted to describe or constitute prior art to the present technology.

Certain medical imaging methods, such as computed tomography (CT) and/or magnetic resonance imaging (MRI), can be used for imaging an anatomy or physiology of a subject. An MRI scanner and/or a CT scanner, for example, can be used to obtain one or more medical images of a subject. In certain scenarios, such as for developing clinical/medical technology, medical images of a plurality of subjects can be made available/accessible to persons other than the subjects themselves. As such, preserving the identity/privacy of said subjects (e.g., within the medical images) becomes an important consideration.

### SUMMARY

This Summary is provided to introduce a selection of concepts in a simplified form that are further described below in the Detailed Description. This Summary is not intended to identify key features or essential features, nor is it intended to limit the scope of the claims included herewith.

The present disclosure is directed towards systems and methods for anonymizing and/or de-identifying an image of a subject by rendering a distinguishing feature of a subject (e.g., an identifying physiological and/or anatomical characteristic) indistinguishable (e.g., by obscuring, obfuscating, concealing, and/or modifying the distinguishing feature in the image). The systems and methods, for instance, can anonymize a CT image, a MR image, a positron emission tomography (PET) image, and/or other medical images by removing, replacing, and/or covering one or more segments of the image that include one or more distinguishing features of the subject (e.g., a plurality of teeth, one or both eyes, and/or other anatomical or physiological features of the subject). In one example, said systems and methods can include applying one or more filtering operations (e.g., a 3D convolution of a Gaussian filter) to the image segment(s) to anonymize the image.

In one aspect, the present disclosure is directed to a method for anonymizing image data (e.g., medical images, such as a computed tomography (CT) image and/or a magnetic resonance imaging (MRI) image) by modifying the image to render a distinguishing feature indistinguishable/unidentifiable. A computing system may obtain a medical image of a subject. The medical image may comprise a set of slices and be associated with a set of metadata regarding the medical image and the subject. In certain embodiments, each slice of the set of slices can be a medical image, wherein the set of slices may be combined to generate a second medical image. In some embodiments, the medical image(s) (and/or other types of images) can be of one or more types, such as a Digital Imaging and Communications in Medicine (DICOM) image, a JPEG (or other types of lossy compression) image, a Portable Network Graphics (or other types of lossless compression) image, and/or other image types/formats. The computing system may identify, based on the set of metadata, one or more regions of interest (ROIs) of the subject in the medical image. The ROIs may correspond with a condition to be evaluated by a clinician using the medical image. The computing system may select, based on the ROIs, a modification technique to apply to the medical image. Selecting the modification technique may comprise determining an image segment that is situated outside of the identified ROIs. The image segment may comprise a distinguishing feature of the subject. The computing system may generate a modified image by applying the selected modification technique to the medical image to modify the set of slices or a subset thereof to thereby render the distinguishing feature in the image segment indistinguishable. The computing system may perform an operation using the modified image. The operation may comprise at least one of (1) transmitting the modified image to another computing system, (2) displaying the modified image on a display screen, or (3) storing the modified image in a non-volatile computer-readable storage medium of the computing system.

In certain embodiments, the medical image can be a volume rendering of the set of slices. The subject can be identifiable based on the distinguishing feature as a result of the volume rendering. In some embodiments, obtaining the medical image may comprise applying a volume rendering technique to the set of slices to generate the medical image. In certain embodiments, obtaining the medical image may comprise using a set of imaging detectors to scan the subject and thereby generate the set of slices. In some embodiments, determining the image segment may comprise detecting the distinguishing feature in the medical image, and delineating the distinguishing feature to encapsulate, in the medical image segment, the distinguishing feature or a portion thereof. In certain embodiments, determining the image segment may comprise determining an intensity threshold that will identify a contour of the image segment. A filter can be applied to the contour of the medical image segment. In some embodiments, applying the filter may blur the image segment of the medical image.

In some embodiments, determining the image segment may comprise receiving, via a user input, a selection of a boundary of the distinguishing feature. In some embodiments, the selected modification technique may comprise adjusting one or more intensities of pixels in the image segment. Adjusting the one or more intensities of the pixels may comprise changing, to a plurality of intensity values, the intensities of a minimum percentage of pixels in the image segment. In certain embodiments, adjusting the one or more intensities of the pixels may comprise changing the intensities to a maximum intensity of the medical image. In some embodiments, adjusting the one or more intensities of the pixels may comprise changing the intensities to a minimum intensity of the medical image.

In certain embodiments, an anonymization metric can be generated based on application of the modification technique to the medical image. The anonymization metric can be determined to be below a threshold. In response to determining the anonymization metric is below the threshold, a second modification technique can be applied to the medical image. In some embodiments, the image segment may comprise one or both eyes of the subject. In certain embodiments, may comprise a plurality of teeth of the subject. In some embodiments, the image segment may comprise a face of the subject. In certain embodiments, the image segment may comprise a head of the subject. In some embodiments, the distinguishing feature can be an anatomical or physiological abnormality of the subject. In some embodiments, the medical image may be based on a CT scan. In some embodiments, the medical image can be based on a MRI scan.

In one aspect, the present disclosure is directed to a computing system for anonymizing image data by modifying the image to render a distinguishing feature indistinguishable. The computing system may comprise one or more processors and a non-transitory computer-readable medium having instructions stored thereon. The one or more processors can execute the instructions stored on the non-transitory computer-readable medium. Upon execution of the instructions by the one or more processors, the instructions may cause the computing system to obtain, by the one or more processors, a medical image of a subject. The medical image may comprise a set of slices and be associated with a set of metadata regarding the medical image and the subject. The instructions may cause the computing system to identify, by the one or more processors, one or more regions of interest (ROIs) of the subject in the medical image based on the set of metadata. The ROIs may correspond with a condition to be evaluated by a clinician using the medical image. The instructions may cause the computing system to select, by the one or more processors, a modification technique to apply to the medical image based on the ROIs. Selecting the modification technique may comprise determining an image segment that is situated outside of the identified ROIs. The image segment may comprise a distinguishing feature of the subject. The instructions may cause the computing system to generate, by the one or more processors, a modified image by applying the selected modification technique to the medical image to modify the set of slices or a subset thereof to thereby render the distinguishing feature in the image segment indistinguishable. The instructions may cause the computing system to perform, by the one or more processors, an operation using the modified image. The operation may comprise at least one of (1) transmitting the modified image to another computing system, (2) displaying the modified image on a display screen, or (3) storing the modified image in a non-volatile computer-readable storage medium of the computing system.

In certain embodiments, the medical image can be a volume rendering of the set of slices. The subject can be identifiable based on the distinguishing feature as a result of the volume rendering. In some embodiments, obtaining the medical image may comprise applying a volume rendering technique to the set of slices to generate the medical image. In certain embodiments, obtaining the medical image may comprise using a set of imaging detectors to scan the subject and thereby generate the set of slices. In some embodiments, determining the image segment may comprise detecting the distinguishing feature in the medical image, and delineating the distinguishing feature to encapsulate, in the medical image segment, the distinguishing feature or a portion thereof. In certain embodiments, determining the image segment may comprise determining an intensity threshold that will identify a contour of the image segment. In some embodiments, the instructions may cause the computing system to apply a filter to the contour of the medical image segment. In some embodiments, applying the filter may blur the image segment of the medical image.

In some embodiments, determining the image segment may comprise receiving, via a user input, a selection of a boundary of the distinguishing feature. In some embodiments, the selected modification technique may comprise adjusting one or more intensities of pixels in the image segment. Adjusting the one or more intensities of the pixels may comprise changing, to a plurality of intensity values, the intensities of a minimum percentage of pixels in the image segment. In certain embodiments, adjusting the one or more intensities of the pixels may comprise changing the intensities to a maximum intensity of the medical image. In some embodiments, adjusting the one or more intensities of the pixels may comprise changing the intensities to a minimum intensity of the medical image.

In certain embodiments, the instructions may cause the computing system to generate an anonymization metric based on application of the modification technique to the medical image. The instructions may further cause the computing system to determine that the anonymization metric is below a threshold. In response to determining the anonymization metric is below the threshold, the instructions may cause the computing system to apply a second modification technique to the medical image. In some embodiments, the image segment may comprise one or both eyes of the subject. In certain embodiments, may comprise a plurality of teeth of the subject. In some embodiments, the image segment may comprise a face of the subject. In certain embodiments, the image segment may comprise a head of the subject. In some embodiments, the distinguishing feature can be an anatomical or physiological abnormality of the subject. In some embodiments, the medical image may be based on a CT scan. In some embodiments, the medical image can be based on a MRI scan.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising a client device in communication with server device.

FIG. 1B is a block diagram depicting a cloud computing environment comprising client device in communication with cloud service providers.

FIGS. 1C and 1D are block diagrams depicting embodiments of computing devices useful in connection with the methods and systems described herein.

FIG. 2 illustrates an example system for implementing the disclosed approach for anonymizing an image of a subject, according to potential embodiments.

FIG. 3 illustrates a flow diagram of an example process for anonymizing an image of a subject, according to potential embodiments.

FIGS. 4A to 4F illustrate example representations of a rendering of a set of slices of a medical image, according to potential embodiments.

FIGS. 5A and 5B illustrate an example approach for determining one or more image segments that are situated outside of one or more ROIs, according to potential embodiments.

FIGS. 6A, 6B, 7, 8A, 8B, 9, 10, 11A and 11B illustrate example approaches for generating a modified image by applying a modification technique to a medical image, according to potential embodiments.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein.

Section B describes embodiments of systems and methods of the present technology for anonymizing an image of a subject by applying one or more modification techniques to the image.

### A. Computing and Network Environment

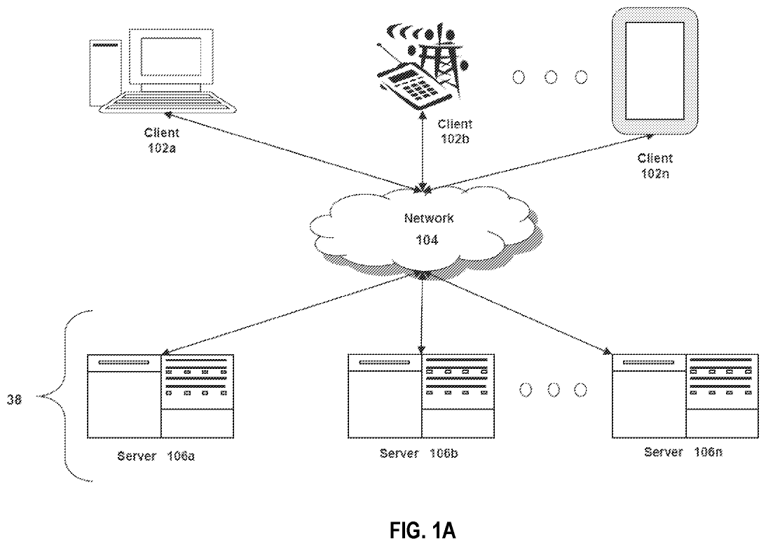

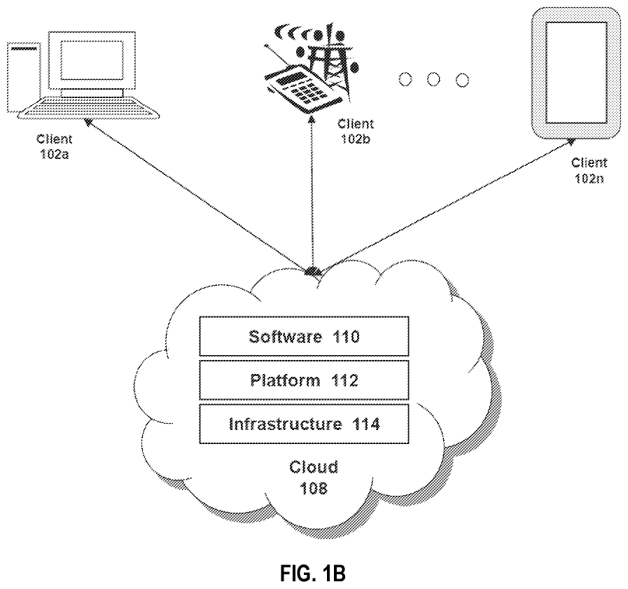

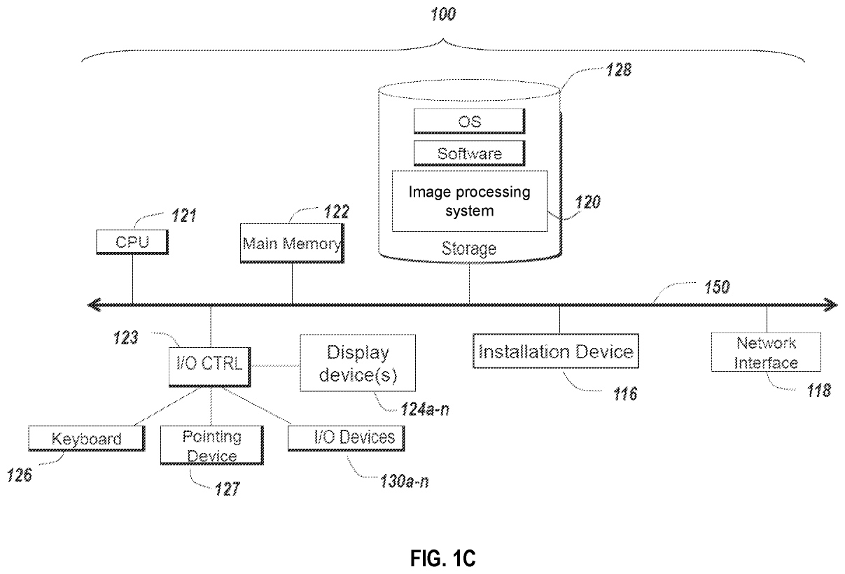

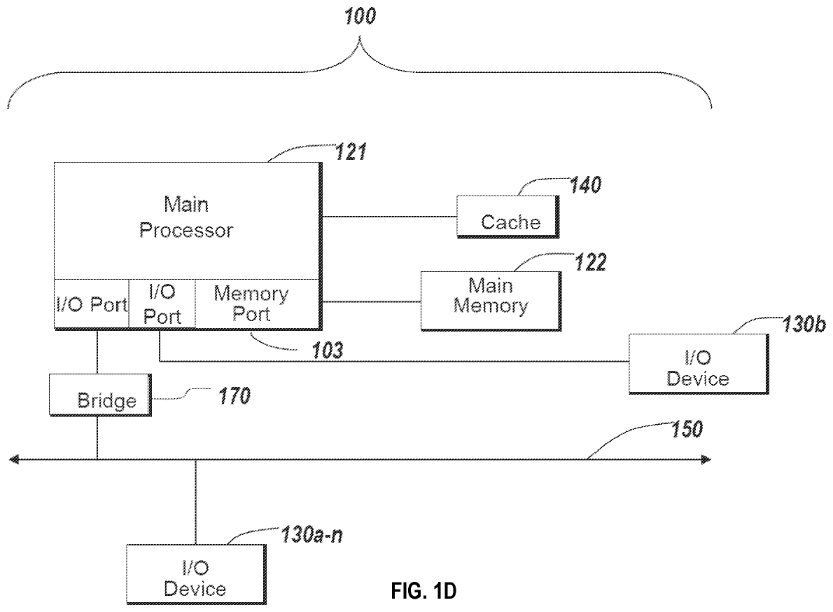

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (Saas) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of an image processing system 120. As shown in FIG. 1D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g. one or more hard disk drives or redundant arrays of independent disks) for storing an operating system or other related software, and for storing application software programs such as any program related to the software for the image processing system 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, WINDOWS 8, and WINDOWS 10 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Systems and Methods for Anonymizing an Image of a Subject

In medical imaging applications (e.g., MRI imaging, CT imaging, PET imaging, and/or other types of medical imaging), radiologists and/or other clinicians can view/analyze one or more medical images of a subject for diagnostic and/or treatment purposes. Although said medical image(s) can be used for said intended purposes, the medical image(s) may contain one or more physiological and/or anatomical characteristics of the subject that can enable the subject to be identified (e.g., based on the medical image(s)) by someone not authorized or otherwise not needing to know the identity of the subject. In certain scenarios (e.g., in applications where large volumes of medical images are accessible to a plurality of people), malicious actors can attempt to apply one or more techniques (e.g., volume rendering of a set of slices) to the medical image(s) in an effort to identify the subject. In many cases, subjects may be identified with no ill intent. As such, identifiable information of the subject within (or associated with) the medical image(s) may be anonymized and/or removed to protect the privacy and/or identity of subjects.

The systems and methods presented herein include a novel approach for anonymizing and/or de-identifying an image of a subject (e.g., making the subject un-identifiable based on the image) by rendering one or more distinguishing features of a subject (e.g., identifying physiological and/or anatomical characteristics) indistinguishable (e.g., by obscuring, obfuscating, concealing, and/or modifying the distinguishing feature(s) in the image). The novel approach can include modifying and/or adjusting a medical image such that the subject depicted within the image is unidentifiable, but the medical image can be utilized for clinical purposes (e.g. for diagnosing, monitoring, evaluating, and/or treating a medical condition). For instance, the systems and methods described herein can use metadata (or other medical/clinical information) to identify one or more ROIs of the subject within the image, wherein the ROIs correspond to a medical condition to be evaluated by a clinician. As such, an area of the image that is outside of the identified ROIs and includes distinguishing feature(s) can be identified, and consequently modified, to anonymize the image while preserving its medical/clinical usability. In various potential embodiments, a set of images obtained previously may be modified retrospectively in batch, or images may be modified individually (e.g., in real-time or near real-time as images are captured or processed). In certain embodiments, an image (or portions thereof) may be tagged for having distinguishing features that are not fully (or partially) modified because the distinguishing features at least partially (or completely) fall within a ROI. Such tagging may be in the form of, for example, metadata that indicates the image has distinguishing features that are at least partially (if not completely) unmodified (i.e., un-anonymized), and/or may include an annotation or marking overlaid on the image to demarcate, identify, or otherwise indicate there is a distinguishing feature that is at least partially unmodified and/or partially modified.

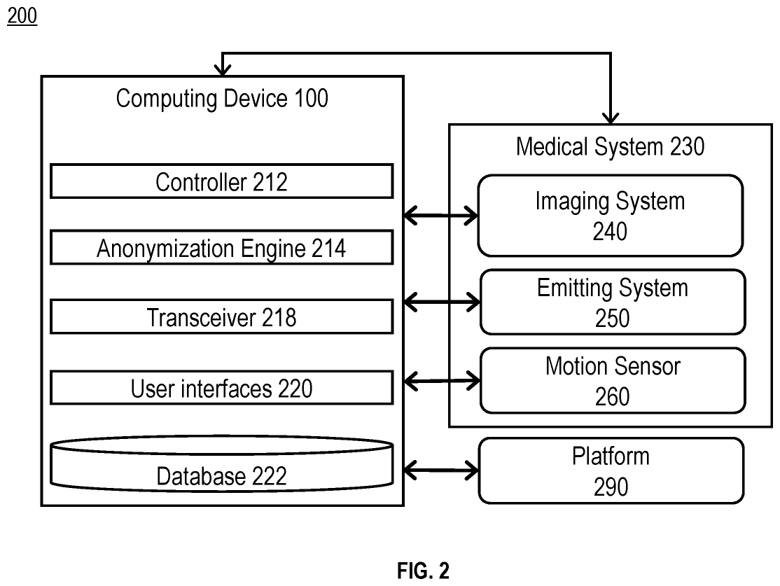

Referring to FIG. 2, in various embodiments, a system 200 may include a computing device 100 (or multiple computing devices, co-located or remote to each other), an imaging system 240 (which may include, e.g., an MRI scanner, a CT scanner, and/or other imaging devices and sensors, such as ray detectors/cameras 245), an emitting system 250 (which may include a ray emission system and/or one or more other devices), and/or a motion sensor 160. In various implementations, the imaging system 240, the emitting system 250, and/or the motion sensor 260 (e.g., to detect motion by a subject) may be integrated into one medical system 230. In certain implementations, computing device 100 (or components thereof) may be integrated with one or more of the medical system 230, the imaging system 240, the emitting system 250, and/or the motion sensor 260. The medical system 230, the imaging system 240, the emitting system 250, and/or the motion sensor 260 may be directed to a platform 290 on which a patient or subject can be situated (so as to image the subject, apply a treatment or therapy to the subject, and/or detect motion by the subject). In various embodiments, the platform 290 may be movable (e.g., using any combination of motors, magnets, etc.) to allow for positioning and repositioning of subjects (such as micro-adjustments due to subject motion).

The computing device 100 (or multiple computing devices) may be used to control and/or receive signals acquired via the imaging system 240, the emitting system 250, and/or the motion sensor 260 directly. In certain implementations, the computing device 100 may be used to control and/or receive signals acquired via the medical system 230. For instance, the computing device 100 may receive and/or obtain one or more medical images of a subject (and/or other information, such as a set of metadata) from the imaging system 240. The computing device 100 may include one or more processors and one or more volatile and non-volatile memories for storing computing code and data that are captured, acquired, recorded, and/or generated (e.g., captured, acquired, recorded and/or generated by the computing device 100 and/or the medical system 230). The computing device 100 may include a controller 212 that is configured to exchange control signals with the medical system 230, the imaging system 240, the emitting system 250, the motion sensor 260, and/or the platform 290, allowing the computing device 100 to be used to control the capture of images (e.g., medical images) and/or signals via the sensors thereof, and position or reposition the subject. The computing device 100 may also include an anonymization engine 214 configured to perform the computations and analyses discussed herein with respect to anonymizing the medical image(s). For example, the anonymization engine 214 can identify one or more regions of interest (ROIs) of the subject in the medical image(s) (e.g., based on a set of metadata associated to the medical images(s) and/or the subject, or based on input by a clinician). In certain embodiments, the anonymization engine 214 may select, identify, and/or determine one or more modification techniques to apply to the medical image based on the identified ROIs. In one example, the anonymization engine 214 may generate one or more modified images by applying the selected modification technique(s) to the medical image(s).

A transceiver 218 allows the computing device 100 to exchange readings, control commands, and/or other data with the medical system 230, the imaging system 240, the emitting system 250, the motion sensor 260, and/or the platform 290 wirelessly or via wires. In one example, the transceiver 218 can allow the computing device 100 to transmit, send, and/or communicate one or more modified image(s) to another computing device. One or more user interfaces 220 (e.g., I/O devices 130) can allow the computing device 100 to receive user inputs (e.g., via a keyboard, touchscreen, microphone, camera, etc.) and provide outputs (e.g., via a display screen, audio speakers, etc.). For instance, the one or more user interfaces 220 can allow the computing device 100 to display and/or present the modified image(s) on a display screen (e.g., a display 124 of the medical system 230 and/or the computing device 100). The computing device 100 may additionally include one or more databases 222 for storing, for example, signals acquired via one or more sensors, signatures, etc. For example, the database(s) 222 may store and/or maintain the medical image(s) and/or the modified medical image(s). In some implementations, database 222 (or portions thereof) may alternatively or additionally be part of another computing device that is co-located or remote and in communication with the computing device 100, the medical system 230, the imaging system 240, the emitting system 250, the motion sensor 260, and/or the platform 290.

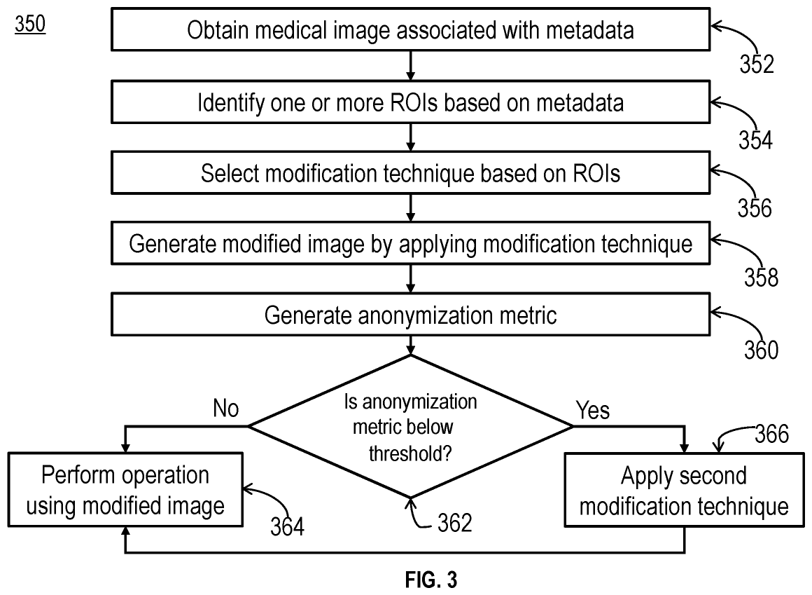

Referring to FIG. 3, depicted is a flow diagram of an embodiment of a method for anonymizing and/or de-identifying an image of a subject by applying one or more modification techniques to the image. The functionalities of the method may be implemented using, or performed by, the components detailed herein in connection with FIGS. 1A-1D and 2. In some embodiments, process 350 can be performed by a client 102 or a server 106. In some embodiments, process 350 can be performed by other entities, such as a computing device 100 and/or a medical system 230 (as discussed in FIGS. 1C-1D and 2). In some embodiments, process 350 may include more, fewer, or different steps than shown in FIG. 3.

In brief overview, process 350 can include obtaining a medical image associated with metadata (352). The process 350 may include identifying one or more ROIs based on metadata (354). The process 350 may include selecting a modification technique based on ROIs (356). The process 350 may include generating a modified image by applying the modification technique (358). The process 350 may include generating an anonymization metric (360). The process 350 may include determining whether an anonymization metric is below a threshold (362). The process 350 may include performing an operation using the modified image (364). The process 350 may include applying a second modification technique (366).

Referring now to operation (352), and in some embodiments, a computing system can obtain, receive, and/or acquire one or more medical images of a subject. The medical image(s) (e.g., MRI image, CT image, PET image, and/or other images) may comprise a set of slices. The medical image(s) can be based on a computed tomography (CT) scan, a magnetic resonance imaging (MRI) scan, and/or other types of scans. In one example, the computing system can receive/obtain the medical image(s) from another system/device, such as a computing device 100 and/or a medical system 230. In certain embodiments, the computing system can obtain the medical image(s) by using a set of imaging detectors (e.g., ray detectors and/or detector coils) to scan/image the subject and thereby generate the set of slices. In some embodiments, the computing system may obtain the medical image(s) by applying a volume rendering technique (e.g., display a 2D projection of 3D sampled image data) to the set of slices to generate the medical image(s). The volume rendering technique can use a set of MRI slices/data (or other types of images), for example, to generate or otherwise provide another image (e.g., a 3D image) that is suitable for facial recognition (e.g., for recognizing or otherwise identifying the subject based on the another image). In some embodiments, one or more volume rendering techniques, such as LIDAR, backscatter X-ray systems (e.g., low-energy backscatter X-ray scanner), and/or other techniques for generating 3D images, can generate or otherwise provide an image (e.g., a 3D image) that is suitable for anonymization, according to the systems and methods discussed herein. As such, any image that is suitable for 3D rendering (e.g., 3D renders of circuits, 3D renders of geographic features, and/or other types of 3D renders) can be anonymized based on the systems and methods described herein. In certain embodiments, the medical image(s) can be a volume rendering of the set of slices. In certain embodiments, the subject can be identifiable (e.g., based on one or more distinguishing features) as a result of volume rendering (e.g., volume rendering a set of slices). In certain embodiments, the subject can be identifiable as a result of generating an isosurface, a meshgrid, and/or other types of surfaces.

In certain embodiments, the medical image(s) can be associated with a set of metadata regarding the medical image(s) and/or the subject (e.g. information describing the medical image(s), such as image-related information for clinical purposes). The set of metadata may include at least one of: matrix dimensions of the image, spatial resolution of the image, pixel depth of the image, photometric interpretation of the image, information about how the image was produced (e.g., timing information, flip angle, number of acquisitions, and/or other information), information about a pharmaceutical injected to the subject, information of the subject (e.g., weight of the subject, diagnosis of the subject, and/or ROIs of the subject in the image), and/or other types of information.

Referring now to operation (354), and in some embodiments, the computing system may identify and/or determine one or more ROIs of the subject in the medical image(s). In certain embodiments, the computing system may identify and/or determine one or more masking portions of the subject in the medical image(s). The ROIs (and/or the masking portions) may correspond with (or be associated with) a condition to be evaluated by a clinician using the medical image(s). In one example, the computing system can identify the ROI(s) (and/or the masking portions) based on (or by using) the set of metadata and/or other information associated with (or included in) the medical image(s). For instance, the set of metadata can include and/or provide information regarding a medical condition/diagnosis of a subject, wherein the medical condition is associated with a particular anatomical feature of the subject. As such, and based on the metadata, the computing system can determine that the ROI(s) include or correspond to one or more regions of the medical image that include the particular anatomical feature of the subject (e.g., associated with the medical condition).

Referring now to operation (356), and in some embodiments, the computing system may select and/or identify a modification technique to apply to the medical image(s). In one example, the computing system may select one or more modification techniques to apply to the medical image(s) (e.g., for anonymizing the medical image(s)). In certain embodiments, a selected modification technique may include or correspond to adjusting, modifying, and/or altering one or more intensities of pixels in an image segment (e.g., image segment outside of the identified ROIs). For example, the image segment may include a set of pixels (e.g., pixels in an image segment), the set of pixels comprising one or more intensities. A selected modification technique can include adjusting, by the computing system, the intensity of each pixel within the set of pixels to another pixel intensity. In one example, the computing system may adjust the one or more intensities of the pixels (e.g., pixels in an image segment) by changing the intensities of a minimum percentage of pixels (e.g., a portion of pixels) in the image segment. For instance, the computing system can change the intensities of the minimum percentage of pixels to a plurality of intensity values. In one example, the computing system may change the intensity of each pixel within the minimum percentage of pixels to an arbitrary intensity value, such that the intensity of each pixel is changed to a separate/distinct intensity value (e.g., arbitrarily varying the intensities of the pixels within the minimum percentage according to white noise, for instance). For instance, the computing system may adjust the intensity of each pixel within the minimum percentage of pixels to a random intensity value, such that a randomization operation (e.g., a randomization of the pixel intensity values) is performed across said pixels.

In certain embodiments, the computing system may adjust the one or more intensities of the pixels by changing the intensities of the pixels in the image segment to a maximum intensity of the medical image. In one example, the computing system may determine and/or identify the maximum intensity across the pixels in the image segment (or other regions of the medical image). Responsive to identifying the maximum intensity, the computing system can change or adapt (e.g., fine-tune to a particular intensity) the intensities of the pixels in the image segment to the determined maximum intensity (e.g., obscuring and/or blocking a distinguishing feature of the subject). In certain embodiments, the computing system may adjust the one or more intensities of the pixels by changing the intensities of the pixels in the image segment to a minimum intensity of the medical image. In one example, the computing system may determine and/or identify the minimum intensity across the pixels in the image segment (or other regions of the medical image). Responsive to identifying the minimum intensity, the computing system can change/adapt the intensities of the pixels in the image segment to the determined minimum intensity (e.g., removing a distinguishing feature of the subject).

In certain embodiments, the computing system may select the modification technique(s) (e.g., to apply to the medical image(s) for anonymization) based on the ROIs. For instance, selecting the modification technique(s) may comprise determining an image segment that is situated outside of the identified ROIs (e.g., an image segment that is not associated with a condition to be evaluated by a clinician). For instance, as discussed above, the computing system can use the set of metadata to determine that the ROI(s) include or correspond to one or more regions of the medical image that include a particular anatomical feature of the subject (e.g., associated with the medical condition). As such, according to the set of metadata for example, the computing system can determine that the image segment includes or corresponds to an area or region of the medical image that excludes the particular anatomical feature of the subject. In certain embodiments, the image segment may include one or more distinguishing features of the subject (e.g., anatomical and/or physiological features that allow identification of the subject). In one example, the image segment may include at least one of: one or both eyes of the subject, a plurality of teeth of the subject, a face of the subject, a head of the subject, and/or other features of the subject. In some embodiments, the distinguishing feature can include or correspond to an anatomical or physiological abnormality of the subject. In certain embodiments, a feature in an image that represents a physiological function (e.g., restricted blood flow) in a region, even if the corresponding region would otherwise not be usable to identify the subject based on anatomically-distinguishing features, may be modified for anonymization purposes if that feature could (e.g., in combination with other information) be used to identify the subject (e.g., using information on a medical condition of the subject, such as an arterial blockage).

In some embodiments, the computing system may determine the image segment by detecting and/or identifying the distinguishing feature in the medical image. For instance, the computing system may detect one or more distinguishing features in the medical image based on (or by using) the set of metadata, the ROIs, image feature recognition techniques, and/or other information/approaches. In certain embodiments, the computing system may detect distinguishing feature(s) according to (or based on) a predetermined set of features expected to be distinguishing (e.g., certain facial features such as nose, teeth, and/or eyes). In one example, said predetermined list can include one or both eyes and a plurality of teeth. As such, the computing system may detect and/or identify the features of the predetermined set (e.g., the eye(s) and the plurality of teeth) in one or more medical images. In some embodiments, the computing system may determine a level to which one or more features of a particular subject can distinguish or identify the subject. For example, the computing system may determine whether a feature of a patient increases the likelihood of being identifiable above a threshold (e.g., 25% or 50% likelihood of being identifiable) for being outside of a normal range or otherwise unusual, such as missing, enlarged, shrunken, or deformed features (e.g., a number of teeth significantly lower than would be expected, and/or other anatomical or physiological features of the subject that are unique based on gender, height, and/or age of the patient). In some embodiments, the likelihood of being identifiable may be based on a comparison of features of the patient with “standard” or “model” features to determine, for each feature, a metric corresponding to deviation from the standard or model feature, with a deviation metric above a deviation threshold rendering a feature as being distinguishing and thus potentially to be modified (e.g., if not within a ROI). In certain embodiments, the computing system may identify the features that result in the subject being too likely to be identifiable as being distinguishing features. Responsive to detecting the distinguishing feature in the medical image, the computing system can delineate, outline, trace, and/or delimit the distinguishing feature. By delineating the distinguishing feature, the computing system may encapsulate or otherwise contain, in the medical image segment, the distinguishing feature or a portion thereof. In some embodiments, the distinguishing feature(s) can be delineated according to automated image recognition techniques and/or a manual user input. In some embodiments, the delineation of the distinguishing feature(s) can be specific to the outline of the distinguished feature(s) (e.g. one or both eyes and/or a plurality of teeth).

In some embodiments, determining the image segment may comprise determining an intensity threshold (e.g., preconfigured threshold and/or a predetermined threshold) that will identify a contour or outline of the image segment. For example, the computing system may identify the contour of the image segment based on a gradient of the pixel intensities. If said gradient meets or exceeds the intensity threshold, the computing system can determine that the image segment has been identified (e.g., the outline of the image segment). In certain embodiments, the computing system may determine the image segment based on a received user input. For instance, the computing system can receive, via user input, a selection of a boundary, contour, and/or outline of the distinguishing feature (e.g., a boundary encapsulating the distinguishing feature). Based on the received selection of the boundary, the computing system may determine the image segment (e.g., the image segment corresponds to the selected boundary). Responsive to identifying the image segment, the computing system can select and/or apply a modification technique (e.g., a filtering operation and/or other image modification techniques) to the image segment (or to the contour of the image segment). For example, the computing system can apply a filter (e.g., a 3D convolution of a Gaussian and/or other types of filters) to the contour of the medical image segment. In certain embodiments, the computing system can apply an image/surface distortion technique to the contour of the medical image segment (e.g. to obscure the medical image segment). Responsive to applying a filter (and/or other modification techniques, such as image distortion), the computing system may blur, obfuscate, and/or obscure the image segment, thereby rendering the subject indistinguishable based on the medical image. In certain embodiments, and in response to applying the filter, the computing system may adjust the intensity value (e.g. add randomization to the intensity values) of each pixel of the contour of the medical image segment to an arbitrary value (e.g., to prevent deconvolution of a performed filtering operation).

Referring now to operation (358), and in some embodiments, the computing system can generate a modified image by applying a selected modification technique to the medical image (e.g., adjusting one or more intensities of pixels in an image segment and/or applying a filtering operation to the image segment) to modify the set of slices or a subset thereof. In certain embodiments, the computing system can apply one or more modification techniques to the medical image. For instance, the computing system can generate a modified image by changing the intensities of a minimum percentage of pixels in the image segment and/or applying a filter to a contour of the image segment. One or more modification techniques may be selected based on various factors, such as the quality, size, type, or purpose of an image, or certain features of the image or portions thereof, such as the type of distinguishing feature, the ratio of the size of the portion of the image that includes distinguishing features relative to the size of the overall image and/or relative to the size of the ROI. For example, using a certain modification technique such as blurring, warping and/or distorting the image, when the image is, for example, of poor quality (e.g., a quality metric such as resolution that is below a quality metric threshold), may make an image more difficult to read by a clinician, as the portion to be examined that is in the ROI may not be as clearly demarcated from the portion that is blurred (or otherwise modified) for having a distinguishing feature; in such cases, a change in intensity level to maximum or minimum levels may better maintain clarity of features to be identified by the clinician. The computing system can additionally or alternatively select one or more modification techniques (e.g., to generate the modified image) based on a computational efficiency or complexity of a particular modification technique (e.g., selecting the most computationally efficient modification technique to minimize or otherwise reduce the amount of time and/or other resources required to perform one or more modifications). Responsive to applying the selected modification techniques, the distinguishing feature (and thereby the subject) can be rendered indistinguishable or otherwise unidentifiable in the medical image.

In some embodiments, the computing system may generate an anonymization metric (360). For instance, the computing system can generate an anonymization metric based on the application of the modification technique to the medical image. In one example, the anonymization metric can include or correspond to a similarity metric. The similarity metric can be used to measure and/or quantify a similarity between a generated modified image (e.g., modified by applying a modification technique) and another image, such as the unmodified medical image and/or other images (or visual representations) of the subject. If a similarity between the modified image and the another image is high (e.g., above a predetermined threshold), the subject can be (or may be deemed to be) identifiable in the modified image. In another example, the anonymization metric may correspond or correlate with a ratio of a distinguishing feature that has been modified, with a certain minimum ratio (e.g., a modification ratio that is at or above a threshold ratio) deemed to be sufficient to render a subject unidentifiable. In some embodiments, the anonymization metric can be used to measure or otherwise quantify a level of anonymity of a subject in the modified image (e.g., determine how unidentifiable or unrecognizable the subject is in the modified image). In certain embodiments, the computing system may determine whether the anonymization metric is below a threshold (362). If the anonymization metric is below the threshold (e.g., the subject remains identifiable in the modified image), the computing system may further apply a second modification technique to the medical image (e.g., to further anonymize/de-identify the subject in the modified image) (366). If, instead, the anonymization metric meets or exceeds the threshold (e.g., the subject is unidentifiable/anonymized in the modified image), the computing system may perform and/or execute an operation using the modified image (364).

Referring now to operation (364), and in some embodiments, the computing system may perform an operation using the modified image. For instance, responsive to generating the image, the computing system can perform one or more operations using the modified image. For example, the computing system may transmit, send, and/or communicate the modified image to another computing system. In another example, the computing system may display, indicate, or otherwise provide the modified image on a display screen. In yet another example, the computing system may store and/or maintain the modified image in a non-volatile computer-readable storage medium of the computing system In yet another example, the computing system may print (or send to a printer for printing) a modified image onto a suitable printing medium for subsequent examination.

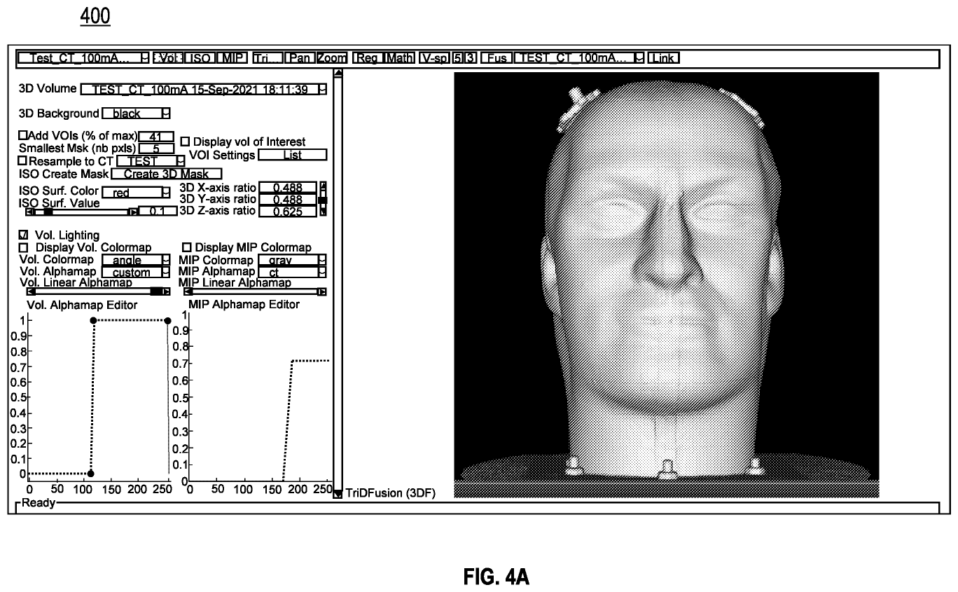

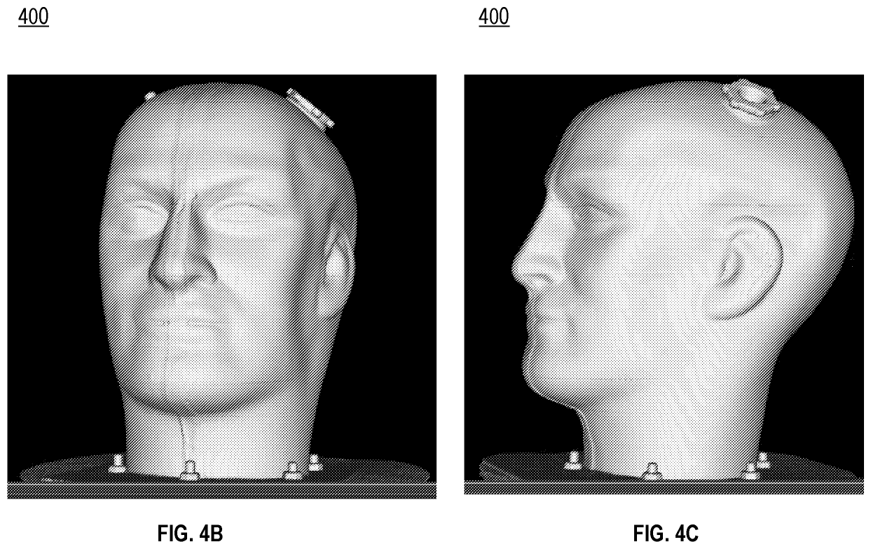

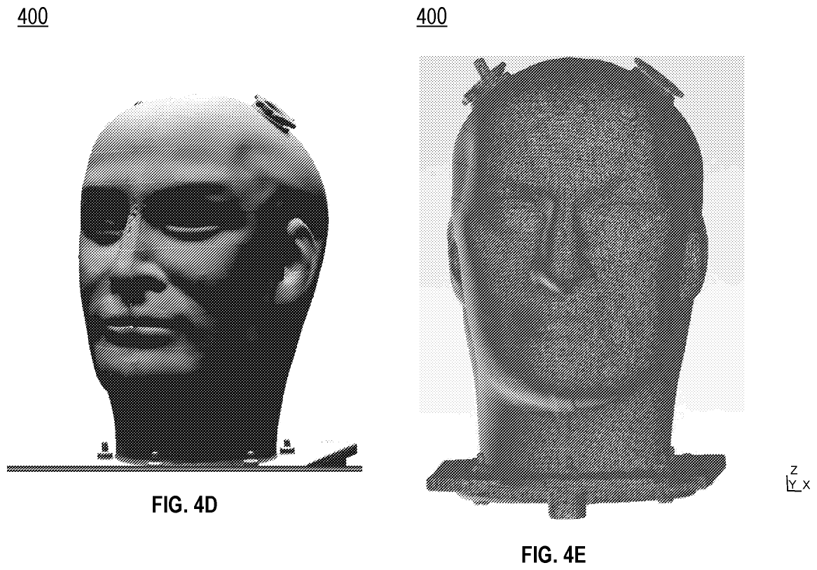

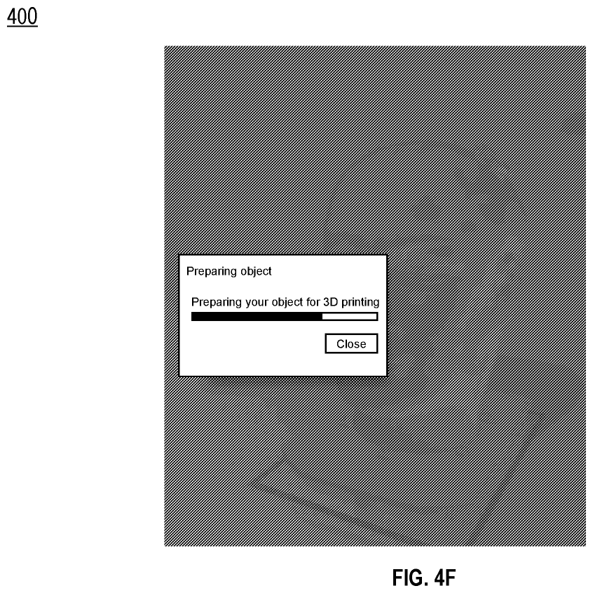

Referring to FIGS. 4A to 4F, depicted are example representations of a volume rendering (e.g., a 3D printing and/or other types of volume rendering) of a set of slices of a medical image (e.g., a medical image of a phantom). As shown in FIGS. 4A to 4C, a subject can become identifiable as a result of the volume rendering. For instance, based on one or more distinguishing features (e.g., one or both eyes, a face, a plurality of teeth, and/or other distinguishing features), a volume rendering of the medical image of the subject can render the subject identifiable. One or more parameters of the volume rendering (e.g., a custom color, an alpha map, and/or other parameters) can be modified or otherwise adjusted to improve or enhance the identifiability of the subject. Referring now to FIGS. 4D to 4E, a subject can become identifiable as a result of applying a volume rendering technique to a set of slices. In FIGS. 4D to 4E, for example, a volume rendering (and/or other types of rendering) can be generated based on a 3D printing of the set of slices, and/or by generating an isosurface (e.g., FIG. 4D), a meshgrid (e.g., FIG. 4E), and/or other types of surfaces.

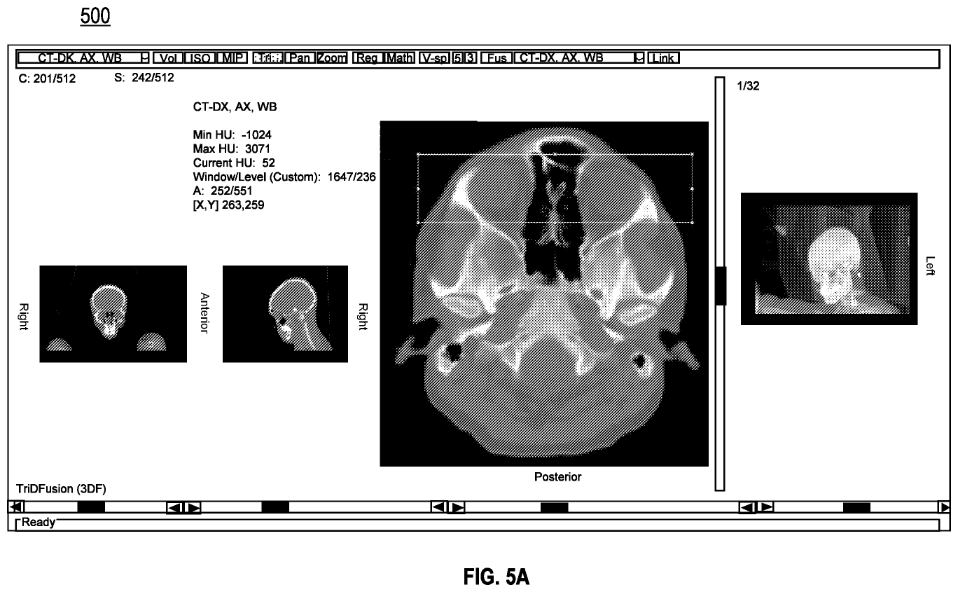

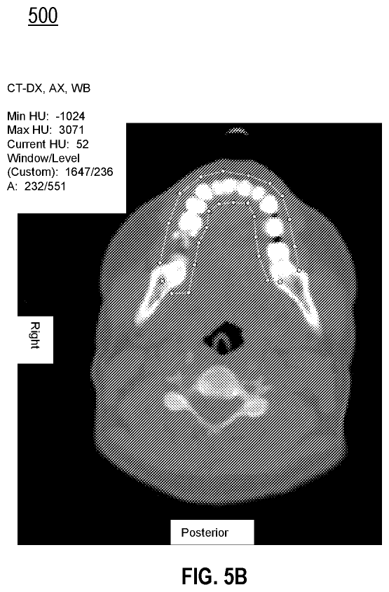

Referring to FIGS. 5A and 5B, depicted is an example approach for determining and/or identifying one or more image segments that are situated outside of one or more ROIs. As seen in FIGS. 5A and 5B, the image segments may comprise a distinguishing feature of the subject, such as a plurality of teeth (FIG. 5B) and/or one or both eyes (FIG. 5A). In certain embodiments, the image segment(s) can be identified across the set of slices of the medical image. The image segment can be determined by delineating the distinguishing feature(s) to encapsulate said feature(s). For example, as depicted in FIGS. 5A and 5B, the plurality of teeth and/or the eye(s) can be identified according to an outline or boundary encapsulating the plurality of teeth and/or the eye(s). The delineation of the distinguishing features can be performed according to a user input (e.g., a manually drawn boundary encapsulating the feature(s)), and/or based on feature detection/recognition techniques.

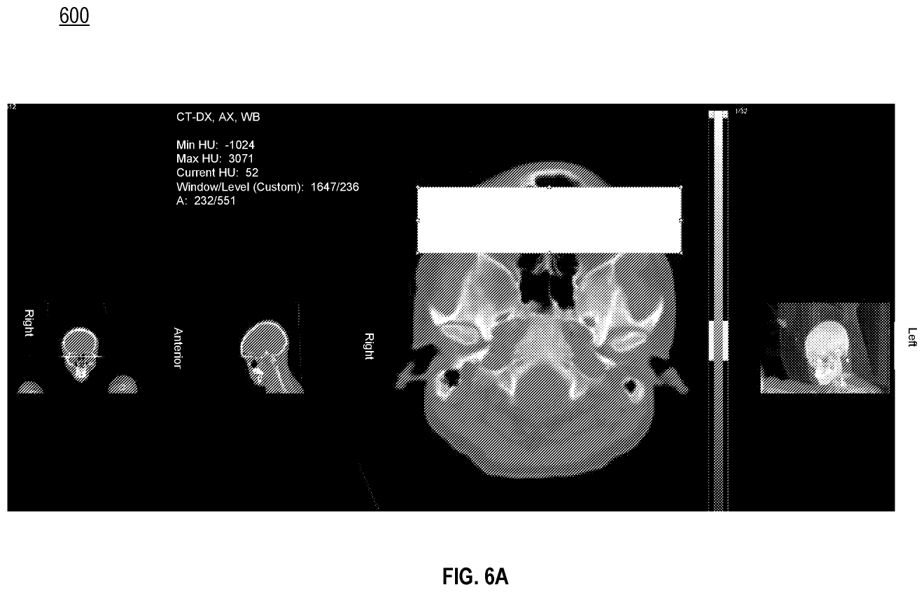

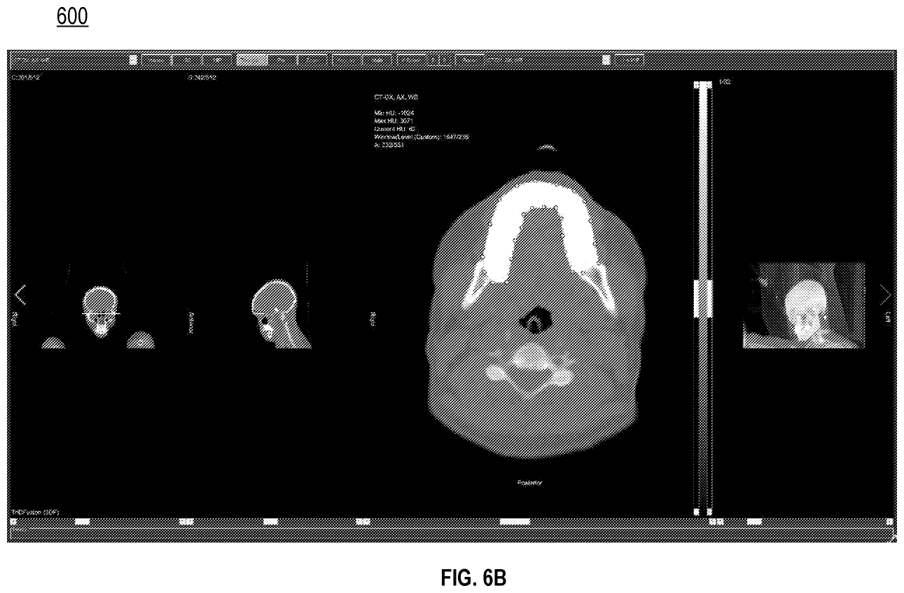

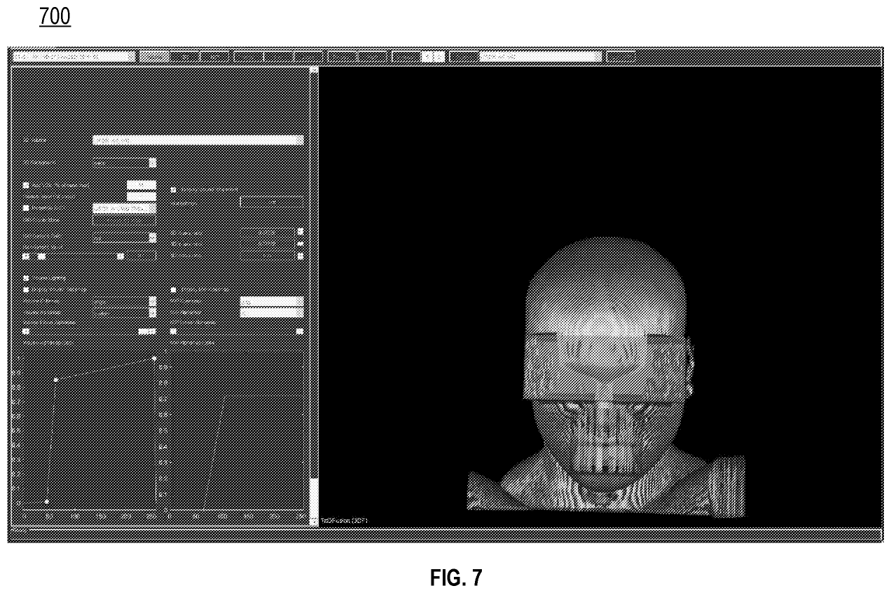

Referring to FIGS. 6A-11, depicted are example approaches for generating a modified image by applying one or more modification techniques to a medical image or portions thereof (e.g., to a subset of slices of the medical image). FIGS. 6A and 6B, for example, illustrate an example approach for adjusting one or more intensities of pixels in one or more image segments (e.g., image segments encapsulating the teeth (FIG. 6B) and/or the eye(s) (FIG. 6A)). For instance, the computing system may adjust the one or more intensities of the pixels by changing the intensities of the pixels in the image segment(s) to a maximum intensity of the medical image (e.g., responsive to identifying the maximum intensity of the medical image). By changing or adjusting the intensities of the pixels to said maximum intensity, the distinguishing feature(s) of the subject (e.g., the teeth and/or the eye(s)) can be obscured, covered, and/or blocked. As such, a volume rendering of the modified set of slices of the medical image (e.g., the slices modified by changing the intensities of the pixels to a maximum intensity) can render the subject indistinguishable or unidentifiable (e.g., the medical image is anonymized), as seen in FIG. 7.

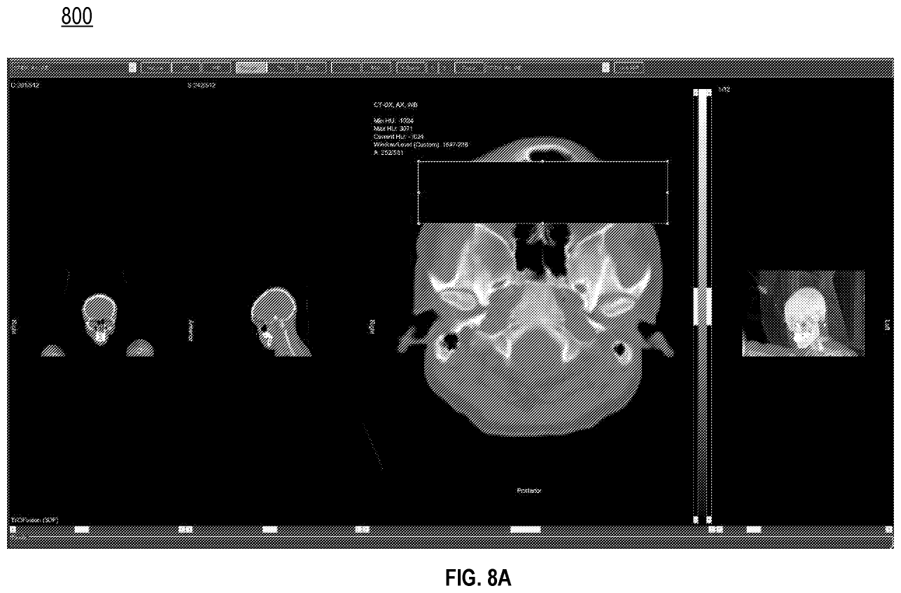

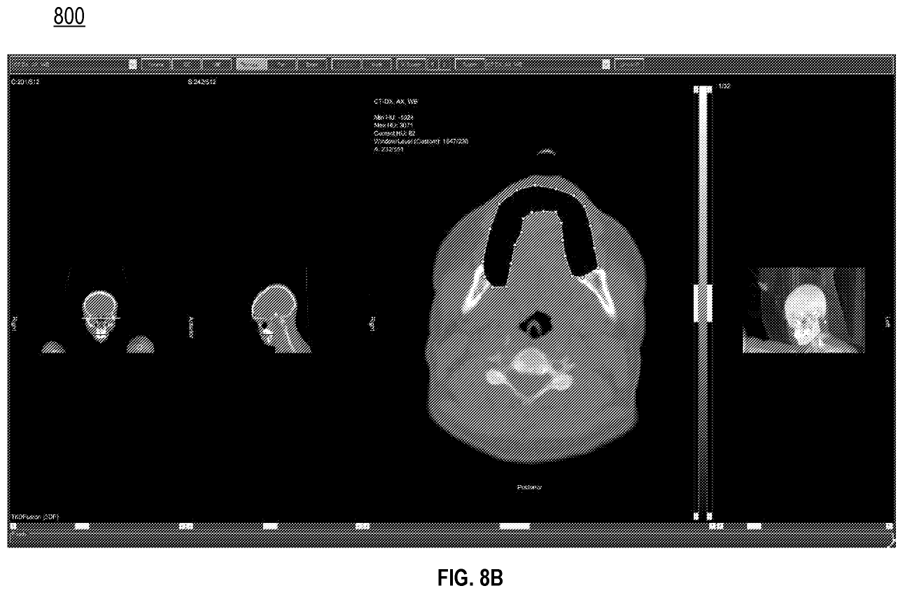

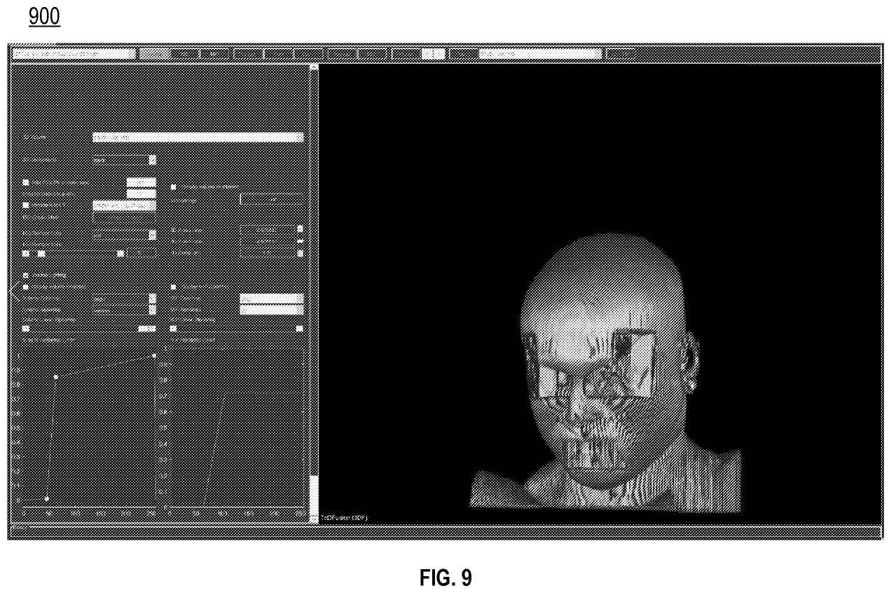

FIGS. 8A and 8B illustrate an example approach for adjusting one or more intensities of pixels in one or more image segments (e.g., encapsulating the teeth (FIG. 8B) and/or the eye(s) (FIG. 8A)). For instance, the computing system may adjust the one or more intensities of the pixels by changing the intensities of the pixels in the image segment(s) to a minimum intensity of the medical image (e.g., responsive to identifying the minimum intensity of the medical image). By changing/adjusting the intensities of the pixels to said minimum intensity, the distinguishing feature(s) of the subject (e.g., the teeth and/or the eye(s)) can be removed and/or extracted from the medical image. As such, a volume rendering of the modified set of slices of the medical image (e.g., the slices modified by changing the intensities of the pixels to a minimum intensity) can render the subject indistinguishable/unidentifiable (e.g., the medical image is anonymized), as seen in FIG. 9.

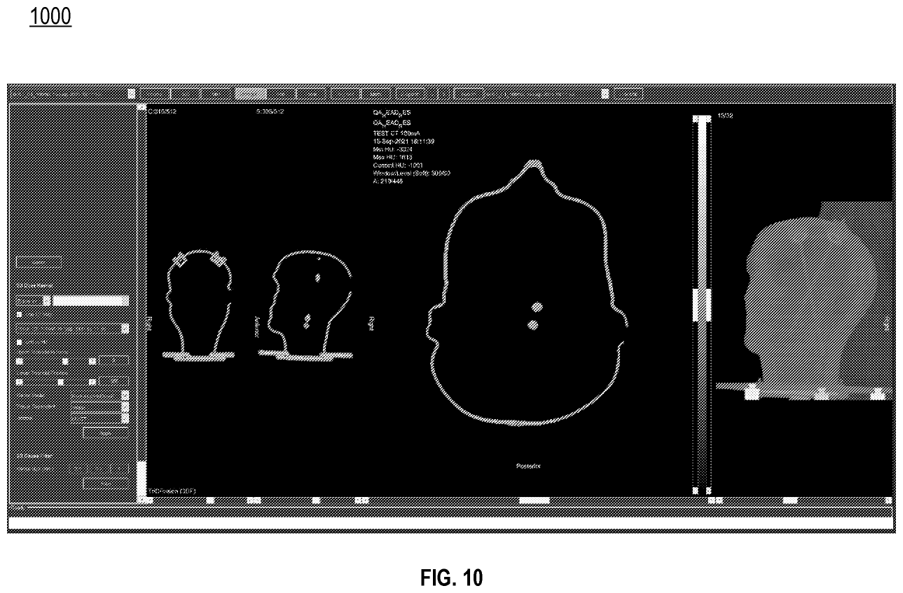

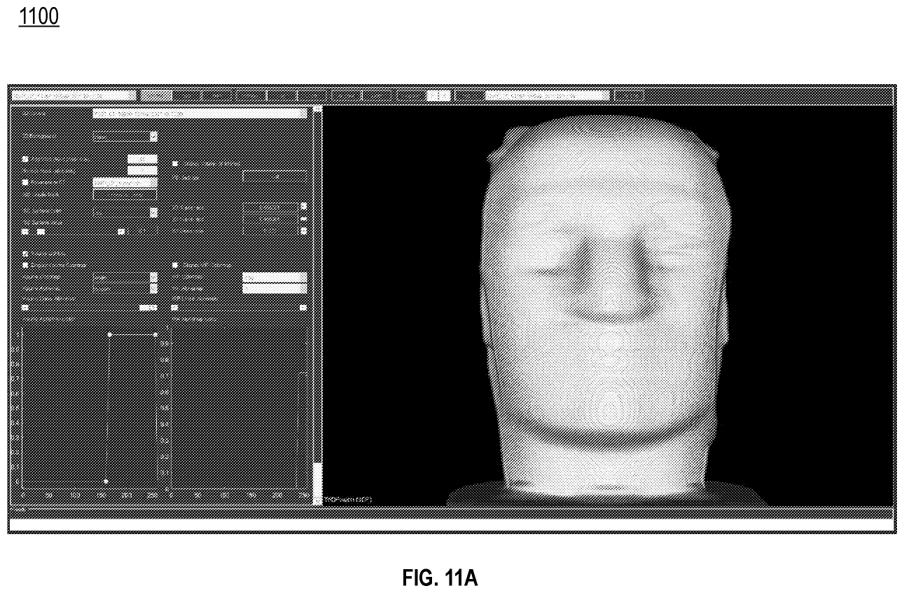

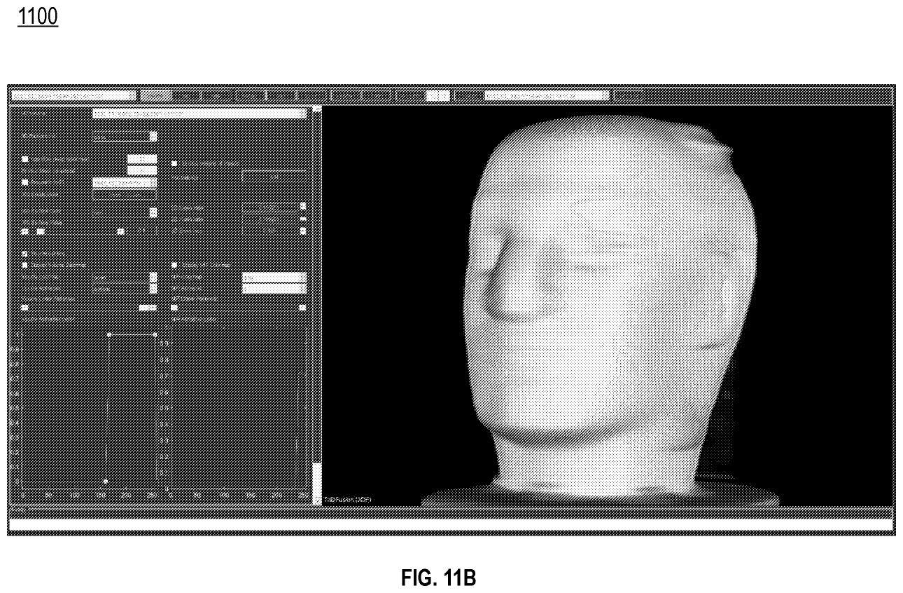

Referring now to FIG. 10, depicted is an example approach for generating a modified image according to an identified contour and/or outline of an image segment comprising a distinguishing feature (e.g., facial features of a subject, such as the chin, nose, lips, eyes, and/or forehead). In one example, the computing system can identify an intensity threshold (e.g., preconfigured threshold and/or a predetermined threshold) that will identify a contour/outline of the image segment. For instance, the computing system may identify the contour of the image segment based on a gradient of the pixel intensities. If said gradient meets or exceeds the intensity threshold, the computing system can determine that the outline of the image segment has been identified/determined. Responsive to identifying the image segment (e.g., the contour of the image segment), the computing system can select and/or apply a filter (and/or other modification techniques) to the contour of the image segment. By applying the filter, the computing system may blur, obfuscate, and/or obscure the contour of the image segment, thereby rendering the subject indistinguishable based on the medical image. FIGS. 11A to 11B, for example, depict example representations of a volume rendering of a modified medical image, wherein a contour of an image segment (e.g., a face of the subject) has been blurred by applying a filter.

It is noted that, in various embodiments, the functions performed by the systems, devices, and components depicted in, for example, FIGS. 1A-1D and 2 may be performed by a greater number of components or fewer components, and may be performed by other combinations of devices and systems. For example, the functions performed by one component as depicted may instead be performed by two or more components, and/or the functions performed by two or more components as depicted may instead be performed by one component. Similarly, functions may be redistributed among components, devices, and systems. For example, the functions performed by one combination of components, devices, and/or systems as depicted may instead be performed by another combination of components, devices, and/or systems.

Various non-limiting example embodiments follow (“Emb”=“Embodiment”):

Emb. A: A method comprising: obtaining, by a computing system, a medical image of a subject, the medical image comprising a set of slices and being associated with a set of metadata regarding the medical image and the subject; identifying, by the computing system, based on the set of metadata, one or more regions of interest (ROIs) of the subject in the medical image, the ROIs corresponding with a condition to be evaluated by a clinician using the medical image; selecting, by the computing system, based on the ROIs, a modification technique to apply to the medical image, wherein selecting the modification technique comprises determining an image segment that is situated outside of the identified ROIs, the image segment comprising a distinguishing feature of the subject; generating, by the computing system, a modified image by applying the selected modification technique to the medical image to modify the set of slices or a subset thereof to thereby render the distinguishing feature in the image segment indistinguishable; and performing, by the computing system, an operation using the modified image, wherein the operation comprises at least one of (1) transmitting the modified image to another computing system, (2) displaying the modified image on a display screen, or (3) storing the modified image in a non-volatile computer-readable storage medium of the computing system.

Emb. B: The method of Emb. A, wherein the medical image is a volume rendering of the set of slices.

Emb. C: The method of either Emb. A or B, wherein the subject is identifiable based on the distinguishing feature as a result of volume rendering.

Emb. D: The method of any of Embs. A-C, wherein obtaining the medical image comprises applying a volume rendering technique to the set of slices to generate the medical image.

Emb. E: The method of any of Embs. A-D, wherein obtaining the medical image comprises using a set of imaging detectors to scan the subject and thereby generate the set of slices.

Emb. F: The method of any of Embs. A-E, wherein determining the image segment comprises detecting the distinguishing feature in the medical image, and delineating the distinguishing feature to encapsulate, in the medical image segment, the distinguishing feature or a portion thereof.

Emb. G: The method of any of Embs. A-F, wherein determining the image segment comprises determining an intensity threshold that will identify a contour of the image segment.

Emb. H: The method of any of Embs. A-G, further comprising applying a filter to a contour of the medical image segment.

Emb. I: The method of any of Embs. A-H, further comprising applying a filter to blur the image segment of the medical image.

Emb. J: The method of any of Embs. A-I, wherein determining the image segment comprises receiving, via a user input, a selection of a boundary of the distinguishing feature.

Emb. K: The method of any of Embs. A-J, wherein the selected modification technique comprises adjusting one or more intensities of pixels in the image segment.

Emb. L: The method of any of Embs. A-K, further comprising changing, to a plurality of intensity values, intensities of a minimum percentage of pixels in the image segment.

Emb. M: The method of any of Embs. A-L, further comprising changing intensities of pixels in the image segment to a maximum intensity of the medical image.

Emb. N: The method of any of Embs. A-M, further comprising changing intensities of pixels in the image segment to a minimum intensity of the medical image.

Emb. O: The method of any of Embs. A-N, further comprising generating an anonymization metric based on application of the modification technique to the medical image.

Emb. P: The method of any of Embs. A-O, further comprising, in response to determining the anonymization metric is below a threshold, applying the first modification technique or a second modification technique to the medical image.

Emb. Q: The method of any of Embs. A-P, wherein the image segment comprises a face of the subject.

Emb. R: The method of any of Embs. A-Q, wherein the image segment comprises one or both facial features of the subject.

Emb. S: The method of any of Embs. A-R, wherein the image segment comprises one or both eyes of the subject.

Emb. T: The method of any of Embs. A-S, wherein the image segment comprises a plurality of teeth of the subject, a bone structure of the subject, and/or a tissue structure of the subject (e.g., cheekbones, a chin, one or more ears, and/or a nose of the subject).

Emb. U: The method of any of Embs. A-T, wherein the image segment comprises a head of the subject.

Emb. V: The method of any of Embs. A-U, wherein the distinguishing feature is an anatomical and/or physiological abnormality of the subject.

Emb. W: The method of any of Embs. A-V, wherein the medical image is based on a computed tomography (CT) scan.

Emb. X: The method of any of Embs. A-W, wherein the medical image is based on a magnetic resonance imaging (MRI) scan.

Emb. AA: A computing system comprising one or more processors configured to: obtain, by the one or more processors, a medical image of a subject, the medical image comprising a set of slices and being associated with a set of metadata regarding the medical image and the subject; identify, by the one or more processors, based on the set of metadata, one or more regions of interest (ROIs) of the subject in the medical image, the ROIs corresponding with a condition to be evaluated by a clinician using the medical image; select, by the one or more processors, based on the ROIs, a modification technique to apply to the medical image, wherein selecting the modification technique comprises determining an image segment that is situated outside of the identified ROIs, the image segment comprising a distinguishing feature of the subject; generate, by the one or more processors, a modified image by applying the selected modification technique to the medical image to modify the set of slices or a subset thereof to thereby render the distinguishing feature in the image segment indistinguishable; and perform, by the one or more processors, an operation using the modified image, wherein the operation comprises at least one of (1) transmitting the modified image to another computing system, (2) displaying the modified image on a display screen, or (3) storing the modified image in a non-volatile computer-readable storage medium of the computing system.

Emb. AB: The computing system of Emb. AA, wherein the medical image is a volume rendering of the set of slices.

Emb. AC: The computing system of either Emb. AA or AB, wherein the subject is identifiable based on the distinguishing feature as a result of the volume rendering.

Emb. AD: The computing system of any of Embs. AA-AC, wherein obtaining the medical image comprises applying a volume rendering technique to the set of slices to generate the medical image.

Emb. AE: The computing system of any of Embs. AA-AD, wherein obtaining the medical image comprises using a set of imaging detectors to scan the subject and thereby generate the set of slices.

Emb. AF: The computing system of any of Embs. AA-AE, wherein determining the image segment comprises detecting the distinguishing feature in the medical image, and delineating the distinguishing feature to encapsulate, in the medical image segment, the distinguishing feature or a portion thereof.

Emb. AG: The computing system of any of Embs. AA-AF, wherein determining the image segment comprises determining an intensity threshold that will identify a contour of the image segment.

Emb. AH: The computing system of any of Embs. AA-AG, the one or more processors further configured to apply a filter to a contour of the medical image segment.

Emb. AI: The computing system of any of Embs. AA-AH, the one or more processors further configured to apply a filter to blur the image segment of the medical image.

Emb. AJ: The computing system of any of Embs. AA-AI, wherein determining the image segment comprises receiving, via a user input, a selection of a boundary of the distinguishing feature.

Emb. AK: The computing system of any of Embs. AA-AJ, wherein the selected modification technique comprises adjusting one or more intensities of pixels in the image segment.

Emb. AL: The computing system of any of Embs. AA-AK, the one or more processors further configured to change, to a plurality of intensity values, intensities of a minimum percentage of pixels in the image segment.

Emb. AM: The computing system of any of Embs. AA-AL, the one or more processors further configured to adjust one or more intensities of one or more pixels to a maximum intensity of the medical image.

Emb. AN: The computing system of any of Embs. AA-AM, the one or more processors further configured to adjust one or more intensities of one or more pixels to a minimum intensity of the medical image.

Emb. AO: The computing system of any of Embs. AA-AN, the one or more processors further configured to generate an anonymization metric based on application of the modification technique to the medical image.

Emb. AP: The computing system of any of Embs. AA-AO, the one or more processors further configured to, in response to determining the anonymization metric is below a threshold, applying the first modification technique or a second modification technique to the medical image.

Emb. AQ: The computing system of any of Embs. AA-AP, wherein the image segment comprises a face of the subject.

Emb. AR: The computing system of any of Embs. AA-AQ, wherein the image segment comprises one or more facial features of the subject.

Emb. AS: The computing system of any of Embs. AA-AR, wherein the image segment comprises one or both eyes of the subject.

Emb. AT: The computing system of any of Embs. AA-AS, wherein the image segment comprises a plurality of teeth of the subject, a bone structure of the subject, and/or a tissue structure of the subject (e.g., cheekbones, a chin, one or more ears, and/or a nose of the subject).

Emb. AU: The computing system of any of Embs. AA-AT, wherein the image segment comprises a head of the subject.

Emb. AV: The computing system of any of Embs. AA-AU, wherein the distinguishing feature is an anatomical and/or physiological abnormality of the subject.

Emb. AW: The computing system of any of Embs. AA-AV, wherein the medical image is based on a computed tomography (CT) scan.

Emb. AX: The computing system of any of Embs. AA-AW, wherein the medical image is based on a magnetic resonance imaging (MRI) scan.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It is noted that terms such as “approximately,” “substantially,” “about,” or the like may be construed, in various embodiments, to allow for insubstantial or otherwise acceptable deviations from specific values. In various embodiments, deviations of 20 percent may be considered insubstantial deviations, while in certain embodiments, deviations of 15 percent may be considered insubstantial deviations, and in other embodiments, deviations of 10 percent may be considered insubstantial deviations, and in some embodiments, deviations of 5 percent may be considered insubstantial deviations. In various embodiments, deviations may be acceptable when they achieve the intended results or advantages, or are otherwise consistent with the spirit or nature of the embodiments.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

1. A method comprising:
obtaining, by a computing system, a medical image of a subject, the medical image comprising a set of slices and being associated with a set of metadata regarding the medical image and the subject;
identifying, by the computing system, based on the set of metadata, one or more regions of interest (ROIs) of the subject in the medical image, the ROIs corresponding with a condition to be evaluated by a clinician using the medical image;
selecting, by the computing system, based on the ROIs, a modification technique to apply to the medical image, wherein selecting the modification technique comprises determining an image segment that is situated outside of the identified ROIs, the image segment comprising a distinguishing feature of the subject;
generating, by the computing system, a modified image by applying the selected modification technique to the medical image to modify the set of slices or a subset thereof to thereby render the distinguishing feature in the image segment indistinguishable; and
performing, by the computing system, an operation using the modified image, wherein the operation comprises at least one of (1) transmitting the modified image to another computing system, (2) displaying the modified image on a display screen, or (3) storing the modified image in a non-volatile computer-readable storage medium of the computing system.

2. The method of claim 1, wherein the medical image is a volume rendering of the set of slices.

3. The method of claim 2, wherein the subject is identifiable based on the distinguishing feature as a result of the volume rendering.

4. The method of claim 2, wherein obtaining the medical image comprises applying a volume rendering technique to the set of slices to generate the medical image.

5. The method of claim 1, wherein obtaining the medical image comprises using a set of imaging detectors to scan the subject and thereby generate the set of slices.

6. The method of claim 1, wherein determining the image segment comprises detecting the distinguishing feature in the medical image, and delineating the distinguishing feature to encapsulate, in the medical image segment, the distinguishing feature or a portion thereof.

7. The method of claim 1, wherein determining the image segment comprises determining an intensity threshold that will identify a contour of the image segment.

8. The method of claim 7, further comprising applying a filter to the contour of the medical image segment.

9. The method of claim 1, wherein determining the image segment comprises receiving, via a user input, a selection of a boundary of the distinguishing feature.

10. The method of claim 1, wherein the selected modification technique comprises adjusting one or more intensities of pixels in the image segment.

11. The method of claim 1, further comprising generating an anonymization metric based on application of the modification technique to the medical image, and in response to determining the anonymization metric is below a threshold, applying the first modification technique or a second modification technique to the medical image.

12. The method of claim 1, wherein the image segment comprises one or more facial features of the subject.

13. The method of claim 1, wherein the distinguishing feature is an anatomical and/or physiological abnormality of the subject.

14. A computing system comprising one or more processors configured to:
obtain a medical image of a subject, the medical image comprising a set of slices and being associated with a set of metadata regarding the medical image and the subject;
identify, based on the set of metadata, one or more regions of interest (ROIs) of the subject in the medical image, the ROIs corresponding with a condition to be evaluated by a clinician using the medical image;
select, based on the ROIs, a modification technique to apply to the medical image, wherein selecting the modification technique comprises determining an image segment that is situated outside of the identified ROIs, the image segment comprising a distinguishing feature of the subject;
generate a modified image by applying the selected modification technique to the medical image to modify the set of slices or a subset thereof to thereby render the distinguishing feature in the image segment indistinguishable; and
perform an operation using the modified image, wherein the operation comprises at least one of (1) transmitting the modified image to another computing system, (2) displaying the modified image on a display screen, or (3) storing the modified image in a non-volatile computer-readable storage medium of the computing system.

15. The computing system of claim 14, wherein the medical image is a volume rendering of the set of slices, and wherein the subject is identifiable based on the distinguishing feature as a result of the volume rendering.

16. The computing system of claim 14, wherein obtaining the medical image comprises at least one of:
applying a volume rendering technique to a set of slices to generate the medical image; or using a set of imaging detectors to scan the subject and thereby generate the set of slices.

17. The computing system of claim 14, wherein determining the image segment comprises detecting the distinguishing feature in the medical image, and delineating the distinguishing feature to encapsulate, in the medical image segment, the distinguishing feature or a portion thereof.

18. The computing system of claim 14, wherein determining the image segment comprises determining an intensity threshold that will identify a contour of the image segment.

19. The computing system of claim 18, the one or more processors further configured to apply a filter to the contour of the medical image segment.

20. The computing system of claim 14, wherein the selected modification technique comprises adjusting one or more intensities of pixels in the image segment.
